(19) United States
(12) Patent Application Publication
(10) Pub. No.: US 2011/0230706 A1
(43) Pub. Date: Sep. 22, 2011

(54) SURGICAL MANIPULATOR

(76) Inventor: Neil Warren, South Glamorgan (GB)

(21) Appl. No.: 13/062,370

(22) PCT Filed: Sep. 4, 2009
(86) PCT No.: PCT/GB2009/051124

§ 371 Date: May 25, 2011

(30) Foreign Application Priority Data
Sep. 5, 2008 (GB) 0816262.0

(51) Int. Cl. A61B 17/00

(52) U.S. Cl. 600/37

(57) ABSTRACT

Surgical Manipulator A surgical manipulator (10) is disclosed which employs a suction cup (30) to hold body parts (50) whilst the manipulator is moved to manipulate the body part (50). Suction at the cup (30) is provided by means of a central passage (16) running through an extension (20) and through a handle part (14) to a vacuum connector (12). The suction at the cup (30) can be adjusted by a slider (15) which adjustably opens the passage (16) to atmosphere. Needles (58) and the like can be introduced into the passage (16) and thereby into the body part (50). For laparoscopic surgery the manipulator can be introduced into a laparoscopic port (52) by collapsing the suction cup (30) within an introducer sleeve (22 FIG. 3). The introducer sleeve (22) can act as a suction device when the cup (30) is collapsed within it. The needle (58) can be replaced by a diathermy electrode (60 FIG. 6) and irrigation is possible using an additional supply tube (121 FIG. 4).

This invention relates to a surgical manipulator, particularly but not exclusively, of the type used during laparoscopic surgery or open surgery.

Conventionally, manipulators used during laparoscopic surgery, and other surgery where remote operation of surgical instruments is employed, have some means of mechanically grasping tissues or body parts, for example jaws, fingers or hooks. Such mechanical means are difficult to use, particularly where fragile body parts are held or manipulated remotely.

Typically, laparoscopic surgical instruments have to be inserted into small ports formed in a body cavity. Usually, these ports are between 5 mm and 12 mm in diameter. Where mechanical manipulators are employed, the jaws etc. are often quite small, so that they can be inserted through the ports. Therefore, the surface area of the mechanical manipulators at their grasping ends tends to be fairly small which causes substantial stress to the body parts at the grasping ends. This can result in inadvertent trauma or tearing of the body part.

Furthermore, large laparoscopic devices are difficult or impossible to insert into a body cavity without making a large incision in the body, thus the inventor has realised that a manipulator is required having a large surface area for gently gripping in use, in addition to the seemingly contradictory need for it to be passed through a small entry port.

The inventor has realised further that there is a need for performing different tasks on, or adjacent the gently gripped area, using the same manipulator that performs the gripping, to reduce time, cost and the number of entry ports.

Embodiments of the invention address the problems mentioned above. According to the invention there is provided a surgical manipulator typically suitable for laparoscopic surgery, the manipulator comprising: a handle; a generally straight tubular extension attached to or forming part of the handle; a flexible suction cup disposed at the distal end of the extension, the cup being in fluid communication with a vacuum passage extending from the cup to the handle, in use the manipulator allowing manipulation of a patient's bodily tissues whilst said tissues are held by means of suction at the cup, characterised in that the handle or the extension further comprises an entrance for a needle or the like arranged so that the needle or the like can be inserted into the extension and exit the manipulator at the suction cup.

In an embodiment the said entrance is a port, openable to allow said needle or the like into the passage, the port having seal means for inhibiting the loss of suction in the passage whilst the port is open to the needle or the like.

Preferably, the seal means includes a replaceable cap, having an elastomeric septum for being pierced by the needle or the like, and providing said inhibiting of the loss of suction by sealing the gap between the needle or the like, and the cap.

In an embodiment, the extension includes an inner needle guide tube for guiding the needle or the like from the entrance or port, to a generally central area of the cup.

In an embodiment the surgical manipulator further comprises a cup compressor having two positions, a first position where the cup is compressed for introducing the cup into a patient's body, and a second position where the cup is released from its compressed state for manipulating use.

Preferably, the cup compressor includes an outer tube slideable on or over the outer surfaces of the extension between the two positions, said sliding being effectible manually from the handle to slide said tube over said cup and into the first cup-compressing position.

More preferably, the outer tube includes an open end into which the cup is collapsed or compressed, the open end being in vacuum communication with said vacuum passage so as to be operable as a suction tool in use when said cup is collapsed or compressed therein.

In an embodiment, adjacent the open end, at least one aperture is formed in the outer tube such that when the open end is closed by bodily tissues or the like, a suction flow is maintainable into the passage via the or each aperture in the tube.

In an embodiment, the vacuum passage is substantially within the extension.

In an embodiment, the passage continues within the handle and terminates at a connector for connecting to a vacuum producing means for providing suction at the cup via the passage.

In an embodiment the passage is openable manually to atmosphere to reduce said suction at the cup.

In an embodiment, the manipulator is connected or connectable to a source of irrigation liquids for delivery at the distal end of the extension.

In an embodiment, a tap is provided for inhibiting the flow of said liquids.

In an embodiment, the handle is in the form of a gun handle with a thumb or finger operated means for said adjusting of the suction at the cup and for operating the tap, and the extension is in the form of a gun barrel having the outer tube slideable thereon and operable by means of a thumb or finger operable trigger adjacent the handle.

In an embodiment, the cup is formed from moulded material such as an elastomer to form a collapsible cup.

In an embodiment, the cup has a bellows arrangement including one or more collapsible bellows.

In an embodiment, the surface of the cup which in use will make contact with said body parts includes one or more cup holes for fluid communication with the passage, the or each cup hole opening into a concavity in the cup, the concavity being formed by a lip piece substantially surrounding the hole or the holes.

In an embodiment the surgical manipulator further comprises an insulated diathermy electrode for passing though the entrance and into the inner guide tube for protruding from the distal end of the extension and for use as a diathermy instrument.

The invention extends to a laparoscopic surgical manipulator including a surgical manipulator as defined above.

The invention extends further to any novel feature described herein or any novel combination of features described herein.

By way of example only, embodiments of the invention will now be described with reference to the drawings wherein:

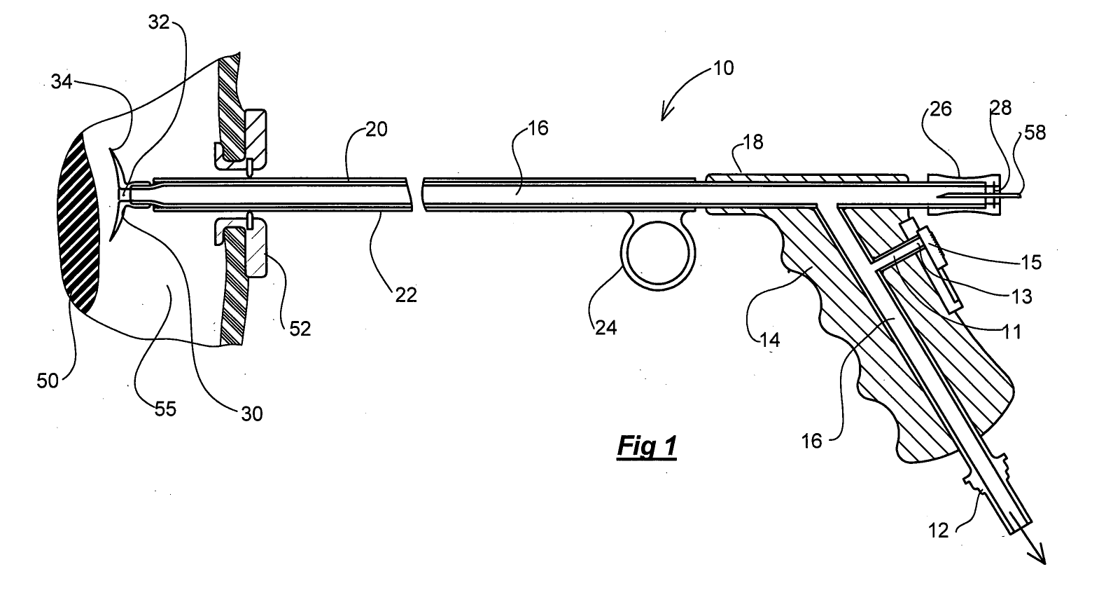

FIG. 1 shows a cross section of a surgical manipulator according to the invention;

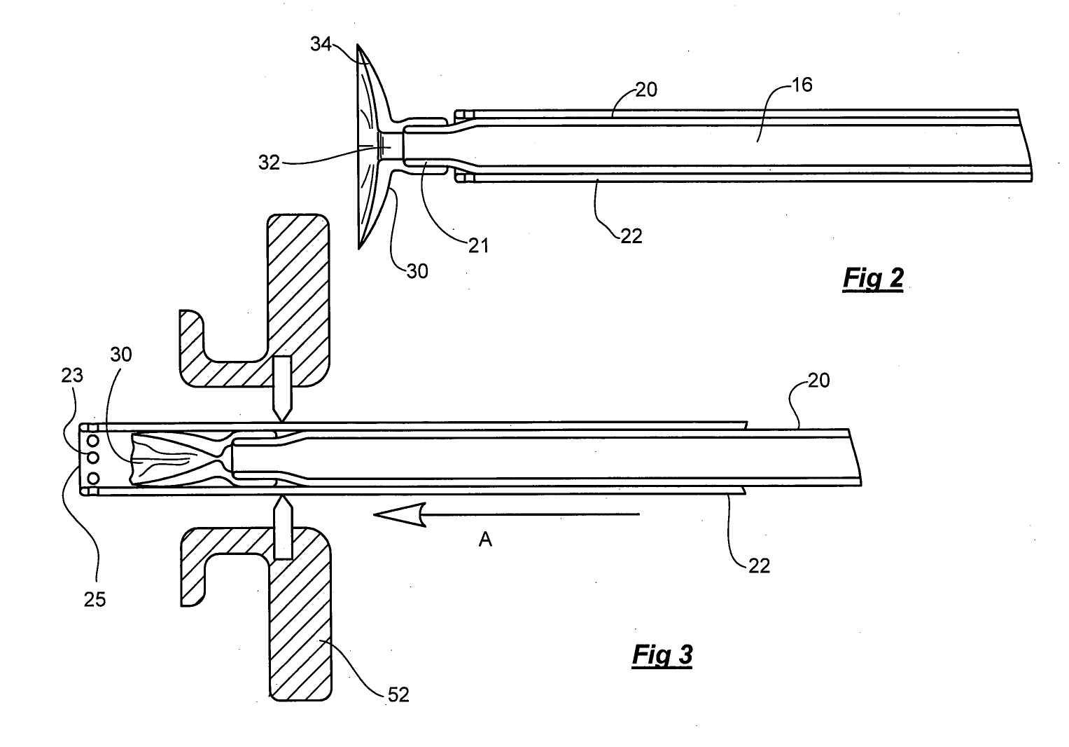

FIGS. 2 and 3 show partial views of the manipulator shown in FIG. 1, in two different configurations.

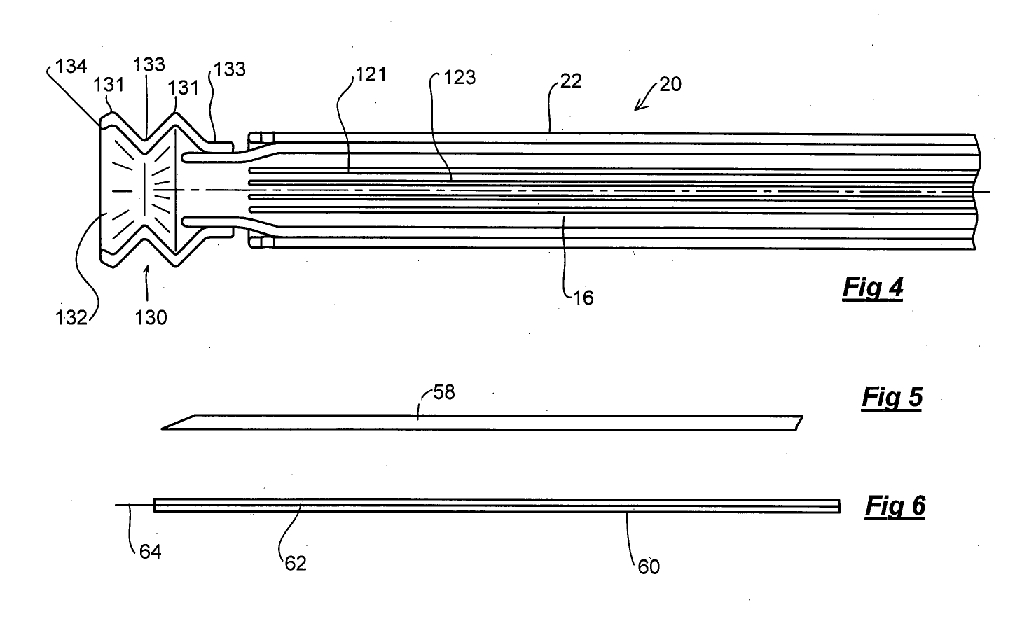

FIG. 4 shows a partial cross section of an alternative embodiment of the invention;

FIGS. 5 and 6 show views of elements for insertion in the embodiment shown in FIG. 4; and

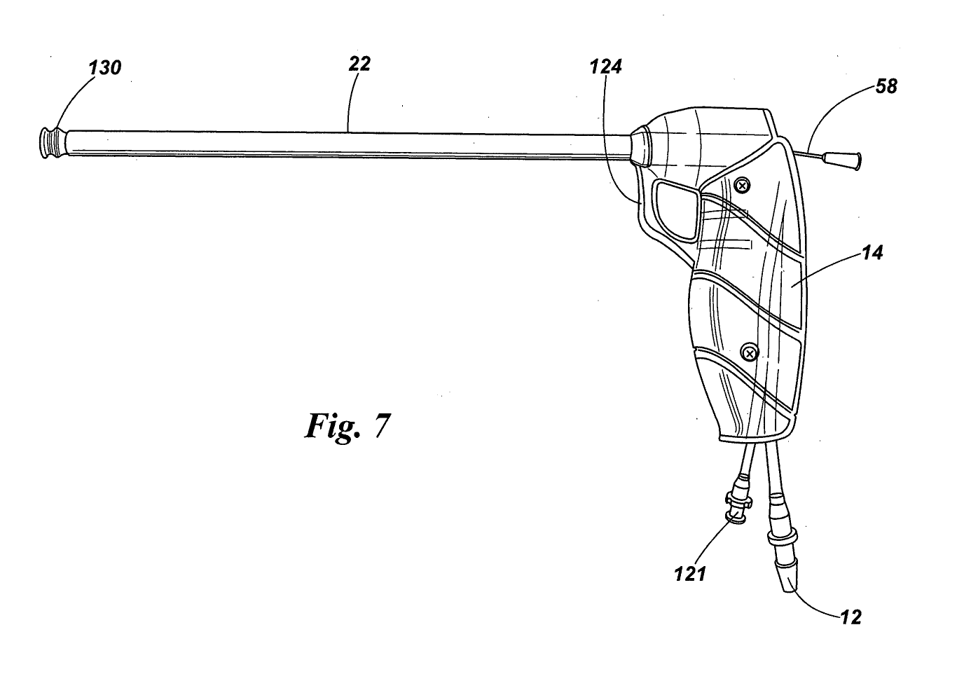

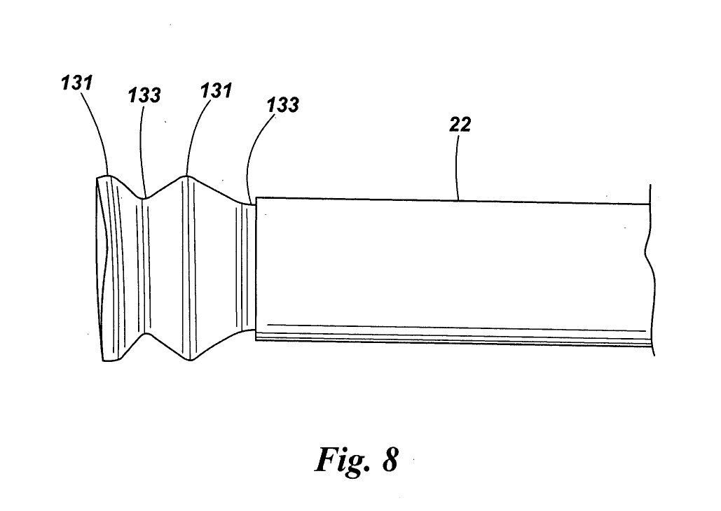

FIGS. 7 and 8 show pictorial views of the alternative embodiment illustrated in FIG. 4.

Referring to FIG. 1 a surgical manipulator 10 is generally shown. The manipulator 10 is used to manipulate body parts 50 within a gas-inflated body cavity 55 of a patient. The manipulator 10 is generally introduced into the cavity 55 through a pre-inserted laparoscopic port 52 with the aid of an introducer 22 described in more detail below. In use, the manipulator 10 is connected to a vacuum source at connector 12, which is located at the base of a manipulator handle 14. The handle 14 has a central passage 16 running from the connector 12 to the top of the handle 18. From the top of the handle 18 the passage 16 extends within an extension piece 20. At the distal end of the extension piece 20 there is a suction cup 30, which includes a central hole 32 in fluid communication with the passage 16. Thus, suction at the cup is generated when the connector 12 is connected to a vacuum source. This suction allows the cup to be held against a body part 50, and the deformation of generally circular flexible lip 34 of the cup 30 maintains the suction effect.

Once the cup 30 is held to the body part 50 the handle 14 can be manipulated so as to pull, push or otherwise manipulate the body part 50 remotely and manually whilst it is held to the suction cup 30.

It may be that the amount of suction at the cup 30 needs to be decreased, in which case a further passage 11 in the handle, which leads to an aperture 13 can be exposed to ambient pressure to increase the pressure in the suction in the passage 16, and thereby reduce the suction at the cup 30. Such a reduction in suction is brought about by moving the slider 15 to expose the aperture 13 to an ambient pressure, using a thumb or finger. Moving the slider 15, so that the opening 13 is completely exposed, will reduce the suction at the cup 30 to such a degree that the body part 50 can be gently released.

To facilitate treatment of body parts 50 a needle or other surgical device 58 (only the tip of which is shown) can be inserted along the extension 20, through the hole 32 in the cup 30 and onto or into the body part 50. The needle can be used for example to assist in the aspiration of cysts, the removal of tissue for a biopsy, for injecting the body part 50 or other surgical procedures. To prevent the reduction of suction in the passage 16 a cap 26 is fitted to the end of the extension 20 behind the handle, and an elastomeric septum 28 seals the passage 16. When a needle 58 or other sharp object pierces the septum 28 the remainder of the septum 28 provides a seal so that suction in the passage 16 is not lost or is not reduced significantly. The septum 28 provides also a self-sealing effect when the needle is removed. To assist a surgeon, the needle 58 includes indicia (not shown) such as graduations or colour coding at its free end to show the depth of penetration past the cup 30.

Referring additionally to FIG. 2, an enlarged view of the distal end of the manipulator 10 is shown. In this view the cup 30 having a central suction hole 32 is more clearly shown. The cup has a generally circular circumference with the outer flexible lip 34 formed from a moulded elastomeric material such as silicon. The lip 34 forms a concavity, which aids the holding of the cup 30 to the body part 50 shown in FIG. 1. The cup 30 is assembled on the extension by pushing it over a reduced end 21 of the extension 20. FIG. 2 also shows the introducer 22.

Referring also to FIG. 3 the extension 20 can be introduced through a port in the laparoscopic port 52 or the like by compressing the cup 30 using the introducer 22. The introducer 22 is a sliding fit over the extension 20 and can be forced over the cup 30 by use of a finger control 24 illustrated in FIG. 1. Movement of the introducer 22 in the direction of arrow A compresses the cup so that the cup is held within the introducer 22, and so it will slide through the port in the body plug 52. The introducer 22 has an open end 25 so that it can be used as a suction tool or implement if required, when the cup is held within the introducer 22. Adjacent the open end 25 is an array of holes 23 which allow suction through those holes even if the open end 25 is blocked by tissue etc.

Referring to FIG. 4 an alternative embodiment is shown which is similar to the embodiment shown in FIG. 1, and where like parts have like reference numerals. In this embodiment, the suction cup 130 is in the form of a bellows arrangement having a concertina of annular ridges 131 and troughs 133 and a lip 134—two ridges a two troughs in this instance, but more, or just one ridge/trough could be used. Further, the extension 20 includes additional concentric tubes 121 and 123. Irrigation tube 121 supplies irrigation fluid to the cup 130 for rinsing functions. A tap (not shown) at the handle can be used to regulate the liquid flow. Inner guide tube 123 is a guide for a needle or other tool. Which can be inserted into the tube at an entrance at the handle, as shown in FIG. 1.

FIG. 5 shows a needle 58 which can be inserted into the inner guide tube 123 in use, for aspirating organs for example by drawing-off fluids, or inserting fluids into an organ. The needle can be inserted while the suction cup 130 is gripping the organ and so there is little chance of fluids leaking into the body cavity while the needle is being used.

FIG. 6 shows a diathermy electrode which is insertable into the inner guide 123, in the same manner as the needle 58. This electrode has an electrically insulated core 62 and an exposed tip 64 which is electrically connected to a high frequency electrical source for cutting tissues in use. The electrode is secured in position in use at the entrance to the inner tube 123 by suitable means. The diathermy electrode could be used in conjunction with the suction cup 130.

FIG. 7 shows a pictorial view of the alternative embodiment. The alternative embodiment is similar in construction to the embodiment shown in FIG. 1 where like parts have like reference numerals. A trigger mechanism 124 is, in this instance, incorporated into the handle 14 and slides the introducer 22 back and forth. A connector 121 is used to supply irrigation liquid to the manipulator.

FIG. 8 shows a pictorial view of the suction cup 130, showing the ridges 131 and troughs 133 mentioned above. As mentioned above the number of folds in the bellows used could be modified, and a detachable suction cup could be used so that different cups could be used for different functions.

Whilst only two embodiments have been described above and illustrated in the drawings, it will be apparent to the skilled addressee that various modifications, alternatives and variations are possible within the scope of the invention. For example, references to vacuum, vacuum means, suction and the like refer to a partial vacuum including gas pressure which is below atmospheric pressure. The manipulator shown is intended to be used for laparoscopic surgery and so needs to be introduced through a port of about 5 to 15 mm in diameter, although smaller and larger ports could be used. The extension of the manipulator is intended to be about 25-35 cm in length, but longer or shorter sizes could be employed, for example shorter manipulators could be used for use with paediatric surgery, or longer ones for surgery on obese patients. An extending extension piece is possible.

Although a generally gun-shaped manipulator device 10 has been shown, it will be apparent that other shapes are possible, for example a bulbous handle at one end of the extension could be used.

A suction cup 30 has been shown having a generally concave working surface although it will be apparent that the cup need not be that shape, for example a generally planar-ended cup could be employed. Although the cap is preferably manufactured from a flexible moulded material, it could be manufactured from any flexible material. The cup 30 preferably has a lip 34 which is of a greater circumference than the extension 20, to afford a relatively large surface area by which to hold body parts 50. However, for intricate surgery, smaller cups could be employed which need not be collapsible if they where to fit within an entry port without being collapsed. For example a cup formed from an ‘O’ ring attached to the distal end of the extension could be used. The introducer 22 is preferably constructed as shown, although, any means of collapsing the cup is possible. The manipulator is shown as an assembly of generally discrete parts, although it could be manufactured such that adjacent discrete parts were moulded integrally, for example, the handle and extension could be formed from one plastics moulding. Although a manually operable manipulator has been described, a machine operable version could be used, for example a robotically operable manipulator is possible, wherein the handle would be a part attachable to a robotic arm or the like.

The embodiment shown provides an atraumatic means of laparoscopically manipulating body parts of humans and animals via a suction cup, with reduced risk of causing inadvertent injury, together with a means for performing actions on the manipulated parts in the sealed environment created by the suction cup when it is used. Other features of the manipulator allow for just one tool to be used where previously numerous tools would have been needed.

1. A surgical manipulator, the manipulator comprising: a handle; a generally straight tubular extension attached to or forming part of the handle; a flexible suction cup disposed at the distal end of the extension, the cup being in fluid communication with a vacuum passage extending from the cup to the handle, in use the manipulator allowing manipulation of a patient's bodily tissues whilst said tissues are held by means of suction at the cup, characterised in that the handle or the extension further comprises an entrance for a needle or the like arranged so that the needle or the like can be inserted into the extension and exit the manipulator at the suction cup.

2. A surgical manipulator as claimed in claim 1 wherein said entrance is a port, openable to allow said needle or the like into the passage, the port having seal means for inhibiting the loss of suction in the passage whilst the port is open to the needle or the like.

3. A surgical manipulator as claimed in claim 2 wherein, the seal means includes a replaceable cap, having an elastomeric septum for being pierced by the needle or the like, and providing said inhibiting of the loss of suction by sealing the gap between the needle or the like, and the cap.

4. A surgical manipulator as claimed in claim 1 wherein, the extension includes an inner needle guide tube for guiding the needle or the like from the entrance or port, to a generally central area of the cup.

5. A surgical manipulator as claimed in claim 1 further comprising a cup compressor having two positions, a first position where
the cup is compressed for introducing the cup into a patient's body, and a second position where the cup is released from its compressed state for manipulating use.

6. A surgical manipulator as claimed in claim 5 wherein, the cup compressor includes an outer tube slideable on or over the outer surfaces of the extension between the two positions, said sliding being effectible manually from the handle to slide said tube over said cup and into the first cup-compressing position.

7. A surgical manipulator as claimed in claim 6 wherein the outer tube includes an open end into which the cup is collapsed or compressed, the open end being in vacuum communication with said vacuum passage so as to be operable as a suction tool in use when said cup is collapsed or compressed therein.

8. A surgical manipulator as claimed in 7 wherein, adjacent the open end, at least one aperture is formed in the outer tube such that when the open end is closed by bodily tissues or the like, a suction flow is maintainable into the passage via the or each aperture in the tube.

9. A surgical manipulator as claimed in claim 1 wherein, the vacuum passage is substantially within the extension.

10. A surgical manipulator as claimed in claim 1 wherein, the passage continues within the handle and terminates at a connector for connecting to a vacuum producing means for providing suction at the cup via the passage.

11. A surgical manipulator as claimed in claim 1 wherein the passage is openable manually to atmosphere to reduce said suction at the cup.

12. A surgical manipulator as claimed in claim 1 wherein, the manipulator is connected or connectable to a source of irrigation liquids for delivery at the distal end of the extension.

13. A surgical manipulator as claimed in claim 12 wherein, a tap is provided for inhibiting the flow of said liquids.

14. A surgical manipulator as claimed in claim 1 wherein, the handle is in the form of a gun handle with a thumb or finger operated means for said adjusting of the suction at the cup and for operating the tap, and the extension is in the form of a gun barrel having the outer tube slideable thereon and operable by means of a thumb or finger operable trigger adjacent the handle.

15. A surgical manipulator as claimed in claim 1 wherein, the cup is formed from moulded material such as an elastomer to form a collapsible cup.

16. A surgical manipulator as claimed in claim 15, wherein the cup has a bellows arrangement including one or more collapsible bellows.

17. A surgical manipulator as claimed in claim 1 wherein, the surface of the cup which in use will make contact with said body parts includes one or more cup holes for fluid communication with the passage, the or each cup hole opening into a concavity in the cup, the concavity being formed by a lip piece substantially surrounding the hole or the holes.

18. A surgical manipulator as claimed in claim 1 further comprising an insulated diathermy electrode for passing though the entrance and into the inner guide tube for protruding from the distal end of the extension and for use as a diathermy instrument.

19. (canceled)

20. A laparoscopic surgical manipulator including a surgical manipulator as defined in claim 1.
